(19) United States
(12) Patent Application Publication
(10) Pub. No.: US 2008/0215132 A1
(43) Pub. Date: Sep. 4, 2008

(54) IMPLANTABLE DEVICES HAVING TEXTURED SURFACES AND METHODS OF FORMING THE SAME

(75) Inventors: S. Eric Ryan, Hopkinton, MA (US); Richard Sahagian, Burlington, MA (US)

Correspondence Address: MILLS & ONELLO LLP, ELEVEN BEACON STREET, SUITE 605, BOSTON, MA, 02108, US

(73) Assignee: CORNOVA, INC., Burlington, MA (US)

(21) Appl. No.: 11/843,402

(22) Filed: Aug. 22, 2007

Related U.S. Application Data
(60) Provisional application No. 60/823,692, filed on Aug. 28, 2006.
(60) Provisional application No. 60/825,434, filed on Sep. 13, 2006.
(60) Provisional application No. 60/895,924, filed on Mar. 20, 2007.
(60) Provisional application No. 60/941,813, filed on Jun. 4, 2007.

(51) Int. Cl. A61F 2/82; A61L 27/04; B31D 3/00

(52) U.S. Cl. 623/1.15; 427/2.25; 623/1.42; 216/56

(57) ABSTRACT

Textured coatings for implantable medical devices can be provided in metallic or polymer surface layers having surface texture factors down to nanometer and sub-nanometer levels. Implantable devices having such textured surfaces and methods of coating such devices comprises providing a substrate surface of an implantable device and forming a coating layer of the substrate surface by directing a mixture of atoms of a first type of material and a second type of material toward the substrate surface while simultaneously and collinearly bombarding the substrate surface with ions, wherein the first type of material is substantially resistant to a removal process and the second type of material is substantially susceptible to a removal process.

### RELATED APPLICATIONS

This application claims the benefit of U.S. Patent Application No. 60/823,692 filed on 28 Aug. 2006, entitled “Adhesive Surfaces for Implanted Devices,” U.S. Patent Application No. 60/825,434 filed on 13 Sep. 2006, entitled “Flexible Expandable Stent,” U.S. patent application Ser. No. 11/613,443 filed on 20 Dec. 2006, entitled “Flexible Expandable Stent,” U.S. Patent Application No. 60/895,924 filed on Mar. 20, 2007, entitled “Implantable Devices and Methods of Forming the Same,” and U.S. Patent Application No. 60/941,813 filed on Jun. 4, 2007 entitled “Implantable Devices Having Textured Surfaces and Method of Forming the Same”, the contents of each being incorporated herein by reference.

This application is related to U.S. Ser. No. ______, filed on or around the filing date of the present application, entitled “Implantable Devices and Methods of Forming the Same,” by Richard Sahagian and S. Eric Ryan, the contents incorporated herein in their entirety by reference.

### FIELD OF THE INVENTION

The present invention relates to implantable biocompatible devices and, in particular, to textured layers for these devices formed with the use of ion beam assisted deposition methods.

### BACKGROUND OF THE INVENTION

Implantable devices provide for the treatment of a myriad of conditions and include devices for heart control and support, muscular-skeletal support, and intravascular support. The surfaces of these devices generally require a significant level of biocompatibility, including stability, smoothness, and resistance to undesired biological interaction. Stents, for example, are implantable prostheses used to maintain and reinforce vascular and endoluminal ducts in order to treat and prevent a variety of cardiovascular conditions. Typical uses include maintaining and supporting coronary arteries after they are opened and unblocked, such as through an angioplasty operation.

As a foreign object inserted into a vessel, a stent can potentially impede the flow of blood. This effect can also be exacerbated by the undesired growth of tissue on and around the stent, potentially leading to complications including thrombosis and restenosis. Typical stents have the basic form of an open-ended tubular element supported by a mesh of thin struts with openings formed between the struts. Designs typically include strong, flexible, and ductile base materials. Stents may also include additional layers to improve radiopacity (such as for positioning with use of a fluoroscope), and layers to increase biocompatibility or provide therapeutic effects in order to promote proper endothelial healing, and/or to resist excessive tissue growth or clotting. Layers for increasing radiopacity may include such materials as gold and may include additional layers of biocompatible and/or therapeutic surface materials such as inert metals and/or polymers with active drug eluting properties.

However, as further described below, traditional techniques of applying these layers to certain substrates fail to adhere them sufficiently to the device, thus creating safety risks which could outweigh the potential benefits. Most stents are manufactured to be reliably deformable in crimped and deployed states. Prior to deployment, a stent is generally in a crimped state and secured about an expandable balloon at the distal end of a catheter. When inserted into position, the balloon and stent are expanded, thus deforming the stent struts and conforming the stent along the inner walls of the vessel. The crimping and expansion process may thus subject any coating materials to additional stresses, increasing the likelihood that the coating undergoes flaking and cracking.

Some metallic materials have been applied onto conventional stents using various techniques including the use of metal bands, electrochemical deposition, and ion beam assisted deposition. However, metal bands are prone to becoming loose, shifting, or otherwise separating from the stent. Moreover, a metal band around a stent can cause abrasions to the intima (i.e., the lining of a vessel wall) during insertion of the device, especially if the bands have sharp edges or outward projections. The physiological response can often be a reclosure of the lumen, thereby negating the beneficial effects of the device. Additionally, cellular debris can be trapped between the intravascular device and the band, and the edges of the band can serve as a site for thrombosis formation.

Electrochemical deposition, including chemical vapor deposition (CVD), physical vapor deposition (PVD), or electroplating, may result in fairly porous stent surface layers, with densities on the order of about 70-75% of full bulk density, or may not provide sufficient adhesion for purposes of medical device applications.

Traditional ion beam assisted deposition (IBAD) of radiopaque materials can be used to improve the adhesion of coatings to the substrate surface. IBAD employs conventional PVD to create a vapor of atoms of, for instance, a noble metal that coats the surface of the substrate, while simultaneously bombarding the substrate surface with ions at energies, typically in the range of 0.8 to 1.5 keV, to impact and condense the metal atoms on the substrate surface. An independent ion source is used as the source of ions.

Coatings produced by IBAD techniques, however, are costly. When evaporating, atoms of expensive noble metals are emitted over a large solid angle compared to that subtended by the device or devices being coated, thus requiring a costly reclaiming process. Moreover, because an evaporator uses a molten metal, it must be located upright on the floor of the deposition chamber to avoid spilling, thereby restricting the size and configuration of the chamber and the devices being coated. Additionally, evaporators cannot deposit mixtures of alloys effectively because of the differences in the alloy components' evaporation rates. As such, the composition of the resulting coating constantly changes.

Furthermore, the conventional IBAD approach is applied by directing the flux of bombarding ions and evaporant, i.e., atoms of metal being deposited, in a non-linear manner, that is, the bombarding ions and metal atoms approach the substrate from different directions. To this end, the energy from the bombarding ions transferred to the evaporant atoms varies depending on the extent to which the two streams overlap. In addition, the number of bombarding ions can be relatively few in number although high in energy, resulting in the metal atoms likely being either implanted tightly into their original impact point or back-sputtering off of the substrate surface. As a result, the growth mechanism of the coating can be inconsistent, and uniform coating properties are difficult to achieve. Moreover, these methods are generally only able to achieve densities of between about 92% to less than 95% of full bulk density.

The above described techniques have been used developed for providing radiopaque surfaces on stents, which enhance the detectability or visualization of what may have been otherwise undetectable core strut materials, and are principally directed toward providing surfaces viewable by fluoroscopes, which requires relatively substantial quantities of radiopaque material, for example, gold, over the substrate surface of the stent, thereby requiring the surfaces to have increased surface dimensions, such as an increased surface area and an increased radiopaque layer thickness generally requiring a thickness greater than 25,000 angstroms. Here, the resulting stent has a larger surface area and is more susceptible to thrombosis or other adverse medical conditions.

Certain core materials including, for example, cobalt-chromium or steel alloys can potentially provide sufficient radiopacity without the need for additional radiopaque layers, however, these materials may lack preferable biocompatibility. The above-described techniques can be used to add coatings with improved biocompatibility, however, with only suboptimal degrees of purity, adhesion, thinness, and/or uniformity of historically preferred biocompatible materials (e.g. titanium, silver, nickel, gold, and platinum) to radiopaque substrate materials.

Various technologies have been developed to provide textured metallic surfaces for promoting bonding with therapeutic agents or other materials. Additional purposes for texturing a surface can include promoting healthy growth and adhesion of endothelial cells about the stent surface. Another purpose can be to provide an increased frictional surface between an implant (e.g. a stent) and delivery system (e.g. balloon catheter) to better secure the implant to the system during delivery such as described in U.S. Pat. No. 6,979,346, incorporated herein by reference. These technologies are also similarly constrained, however, by non-adherent, relatively thick and/or uneven layers with less than optimal biocompatibility over a substrate surface.

### SUMMARY OF THE INVENTION

Embodiments of the present invention are directed to textured outer surfaces for implanted devices and methods of manufacturing the same, which overcome the limitations associated with the abovementioned approaches. In particular, embodiments of the present invention provide improved implantable medical devices with dense, adherent, thin, uniform and biocompatable coatings and methods for their manufacture.

In accordance with embodiments of the invention, devices and methods provide enhanced ion-bombarded textured surface layers on implantable devices suitable for interfacing with internal body tissues. In embodiments of the invention, a micro-textured or nano-textured surface is formed which can promote the smooth passage of blood and optimal growth and attachment of endothelial cells about an outer surface of an implant, thus promoting healthy healing about the implant area. In the case of a stent implant, for example, such healthy healing can maintain the smooth passage of blood. In contrast, an area that hasn't healed smoothly about a stent surface can result in the passage of blood being impeded over the long-term by areas about the stent with very little, or abnormal, growth or by elevated levels of platelet activity which may potentially lead to thrombosis. An overly smooth stent surface may impede attachment of endothelial cells and desired healthy healing, potentially leading to these and other complications.

Additional applications for textured metallic surfaces can provide enhanced adhesiveness with additional layers such as polymers, for providing textured surfaces for the elution of drugs or other agents and/or for increasing friction and improving retention between a stent surface and a delivery system during delivery.

In accordance with an aspect, atoms of a first type of material resistant to a removal process are simultaneously co-deposited with atoms of a second type of material that are not resistant or susceptible to said removal process along with substantially collinearly bombarding ions such as with, for example, one or more magnetrons with unbalanced magnetic fields. The atoms of the first and second types can be controllably deposited in ratios to provide a desired texture. Embodiments include a removal process comprising an electrochemical bath, a first type of material resistant to the bath such as platinum, platinum-iridium, nickel, or alloys and combinations thereof and a second type of material susceptible to the bath such as copper, chromium, silver, or alloys and combinations thereof. After the removal process is applied, a surface having voids (or nucleation sites where the removed atoms were once located) is formed on the surface.

An embodiment includes an implantable device having a capping layer of between 100 and 25000 angstroms with a nano-textured surface. Further embodiments include capping layers with thicknesses of between about 5,000 and 10,000 angstroms and between about 500 and 2500 angstroms. In an embodiment of the invention, the capping layer comprises a biocompatible metal such as, for example, platinum, platinum-iridium, or nickel.

The nano-textured surface can be adapted to provide enhanced adhesiveness to polymers and/or eluting drugs, to provide a surface for facilitating proper re-endothelialization about the surface, to provide a porous surface for eluting drugs, and/or to facilitate improved frictional contact with a deployment system.

For providing a surface to facilitate re-endothelialization, a target surface texture can be fine enough to promote healthy attachment of endothelial cells while avoiding an abundant attraction of unhealthy occlusive cells such as that of platelets, thrombin, and fibrin that could result in, for example, restenosis or thrombosis. In embodiments, the surface has a texture factor (the root mean square of the breadth of the voids) of less than about 3 microns and preferably about 1 micron or less, with embodiments also including surface texture factors of less than 0.01 microns.

An embodiment includes an adhesion layer having one or more parts. An embodiment of a first part of the adhesion layer substantially comprises palladium. In an embodiment of the invention, a second part provides a transition section with a combination comprising platinum and palladium. Embodiments of the transition section include a gradual increase in the concentration of platinum and decrease in the concentration of palladium.

Embodiments provide for thicknesses of between about 0.01 to 0.5 microns for adhesion and/or capping layers, sufficient to provide adequate levels of adhesion between the capping layer and an implant. The adhesion and/or capping layers can have densities of greater than about 95% full bulk density and, in embodiments of the invention, between at least about 95% and 98% full bulk density.

Embodiments include the application of the surfaces on devices that have strong, flexible substrates (e.g. stents) which may undergo flexing during implantation. In an embodiment of the invention, the substrate may include metallic materials such as cobalt chromium or stainless steel. Embodiments of the invention include adhesion layers and/or capping layers between about 0.01 and 0.05 microns in thickness and between about 0.05 and 0.25 microns in thickness.

In accordance with another aspect, the adhesion and capping layers are deposited by ion bombardment methods, an embodiment including generating and directing fluxes of atoms of the implanted materials and bombarding ions onto the target surfaces in a substantially collinear manner. In an embodiment of the invention, the fluxes of atoms and bombarding ions are directed with the use of one or more magnetrons having unbalanced magnetic fields. For generating the transition layers described above, an aspect of the invention provides a method for concurrently depositing atoms in ratios according to the desired mixtures and gradually modifying these ratios during deposition. In an embodiment of the invention, a method is provided for generating the two-part adhesion layer described above, by controllably and concurrently decreasing the content of palladium in the second part of the adhesion layer while gradually increasing the content of platinum in the second part.

In accordance with an aspect, a method is provided for coating an implantable device including the steps of providing a substrate surface of an implantable device, forming a coating layer on the substrate surface by directing a mixture of atoms of a first type of material and of a second type of material toward the substrate surface while simultaneously and substantially collinearly bombarding the substrate surface with ions. The first type of material is substantially resistant to a removal process and the second type of material is substantially susceptible to a removal process. The method further includes the step of creating voids in the coating layer by applying the removal process to selectively remove at least some of the atoms of the second type of material from the coating layer surface.

In an embodiment, the first type of includes at least one of platinum, platinum-iridium, gold, and alloys thereof.

In an embodiment, the second type of material includes at least one of copper, chromium, silver, and alloys thereof.

In an embodiment, the removal process includes exposing the coating layer to an electrochemical bath.

In an embodiment, the removal process includes applying an acid in which the first type of material substantially detaches from the surface of the coating layer and in which the second type of material does not substantially detach from the coating layer. In an embodiment of the invention, the acid includes at least one of at least sulphuric acid, nitric acid, or phosphoric acid.

In an embodiment, the voids of the textured exterior provides a surface texture factor of less than about 3 microns. In an embodiment of the invention, the voids of the textured exterior provide a surface texture factor of less than about 1 micron. In an embodiment, the voids of the textured exterior provide a surface texture factor of less than about 0.1 microns. In an embodiment of the invention, the voids of the textured exterior provide a surface texture factor of less than about 0.01 microns. In an embodiment, the voids of the textured exterior provide a surface texture factor of between about 0.003 and 0.01 microns.

In an embodiment, the deposited mixture of atoms of the first and second types are substantially of a density of greater than about 95% full bulk density.

In an embodiment, the method for coating an implantable device further includes the step of coating the textured exterior with a polymer.

In an embodiment, the method for coating an implantable device further includes the step of infusing the voids of the textured exterior with a therapeutic agent. In an embodiment, the therapeutic agent is at least one of paclitaxel, rapamycin, sirolimus, genes, or stem cells.

In accordance with another aspect, an implantable device is provided including a substrate and one or more coatings on the substrate, the coatings including a textured layer of metal atoms deposited onto the device by a method of deposition with substantially collinear ion-bombardment, the textured layer of metal atoms having a surface factor of less than about 3 microns.

In an embodiment, the exterior of the textured layer has a surface texture factor of less than about 1 micron. In an embodiment, the exterior of the textured layer has a surface texture factor of less than about 0.1 microns. In an embodiment, the exterior of the textured layer has a surface texture factor of less than about 0.01 microns. In an embodiment, the exterior of the textured layer has a surface texture factor of between about 0.001 and 0.01 microns. In an embodiment, the exterior of the textured layer has a surface texture factor of between about 0.0004 and 0.001 microns.

In an embodiment, at least one or more of said coatings is substantially of a density of greater than about 95% full bulk density.

In an embodiment, the implantable device further includes a polymer layer adjacent the textured layer.

In an embodiment, the implantable device further includes a therapeutic agent infused into voids of the textured layer. In an embodiment of the invention, the therapeutic agent is at least one of paclitaxel, rapamycin, sirolimus, dipyridamole, genes, stem cells, and combinations thereof.

In accordance with another aspect, a medical stent is provided including a textured surface with a surface texture factor of less than about 0.01 microns. In an embodiment of the invention, the surface texture factor is between about 0.0004 and 0.01 microns.

In accordance with another aspect, a method for providing a textured polymer surface of an implantable device comprises providing a polymer surface, generating a flux of bombarding ions and directing the ions toward the polymer surface in order to form a plurality of substantially uniformly distributed voids on the polymer surface.

In an embodiment, the ions are noble ions. In an embodiment of the invention, the ions are of at least one of xenon or argon ions.

In an embodiment, the voids provide a surface texture factor of less than about a micron. In an embodiment of the invention, the voids provide a surface texture factor of less than about a nanometer.

In accordance with another aspect, an implantable device is provided having a textured polymer surface formed by a method including the steps of providing a polymer surface, generating a flux of bombarding ions and directing the ions toward the polymer surface in order to form a plurality of substantially uniformly distributed voids on the polymer surface.

In an embodiment, an implantable device having a textured polymer surface is provided having a surface texture factor of less than about 3 microns.

In an embodiment, the textured polymer surface has a surface texture factor of less than about a micron.

### BRIEF DESCRIPTION OF THE DRAWINGS

The structure, operation, and methodology of embodiments of the invention, together with other objects and advantages thereof, may best be understood by reading the following detailed description in connection with the drawings in which each part has an assigned numeral or label that identifies it wherever it appears in the various drawings. The drawings are not necessarily to scale, emphasis instead being placed upon illustrating the principles of the embodiments of the invention. FIGS. 1A-2B are, for example, not intended to accurately reflect relative proportions of layer thicknesses but for illustrating the general order of layer positions.

FIG. 1A is an illustrative cross-sectional view of surface layers on an implantable device prior to texturing in accordance with an embodiment of the invention.

FIG. 1B is an illustrative cross-sectional view of surface layers on an implantable device after texturing in accordance with an embodiment of the invention.

FIG. 1C is an illustrative cross-sectional view of surface layers on an implantable device after texturing in accordance with an embodiment of the invention.

FIG. 1D is an illustrative cross-sectional view of surface layers on an implantable device having a coating over the textured surface in accordance with an embodiment of the invention.

FIG. 2A is an illustrative side view of a stent in accordance with an embodiment of the invention. FIG. 2B is an illustrative transverse cross-sectional view of a strut of the stent of FIG. 2A, taken along section lines I-I′ of FIG. 2A.

FIG. 3 is an illustrative view of an unbalanced magnetron depositing atoms onto a substrate for purposes of forming a textured surface in accordance with an embodiment of the invention.

FIG. 4 is a side-perspective illustrative schematic of an apparatus for coating an implantable device using multiple magnetrons according to an embodiment of the invention.

### DETAILED DESCRIPTION OF EMBODIMENTS OF THE INVENTION

The accompanying drawings are described below, in which example embodiments in accordance with the present invention are shown. Specific structural and functional details disclosed herein are merely representative. The invention may be embodied in many alternative forms and should not be construed as limited to the example embodiments described herein. It will be understood that the drawings are not intended to accurately reflect relative proportions of layer thicknesses but rather to illustrate the general order of layer positions.

Accordingly, specific embodiments are shown by way of example in the drawings. It should be understood, however, that there is no intent to limit the invention to the particular forms disclosed herein, but to the contrary, the invention is to cover all modifications, equivalents, and alternatives falling within the spirit and scope of the claims.

It will be understood that, although the terms first, second, etc. may be used herein to describe various elements, these elements should not be limited by these terms. These terms are used to distinguish one element from another. For example, a first element could be termed a second element, and, similarly, a second element could be termed a first element, without departing from the scope of the present disclosure.

It will be understood that when an element is referred to as being “on,” “adjacent,” “connected to,” or “coupled to” another element, it can be directly on, connected to or coupled to the other element or intervening elements may be present. In contrast, when an element is referred to as being “directly on,” “directly adjacent,” “directly connected to,” or “directly coupled to” another element, there are no intervening elements present. Other words used to describe the relationship between elements should be interpreted in a like fashion (e.g., “between” versus “directly between,” etc.).

It will be understood that the term “directly on,” as used herein, is intended to describe situations where there is a substantial molecular contact between two elements or layers, for example, between an adhesion layer and a substrate, or between a capping layer and a substrate.

It will be understood that the term “gradated mixture,” as used herein, refers to a layer having a composition gradiant comprising a mixture of at least first and second materials, wherein there is a smooth, continuous composition gradient from one side of the layer to the other side such that the ratio of first material to second material is relatively higher at one side and lower at the other side.

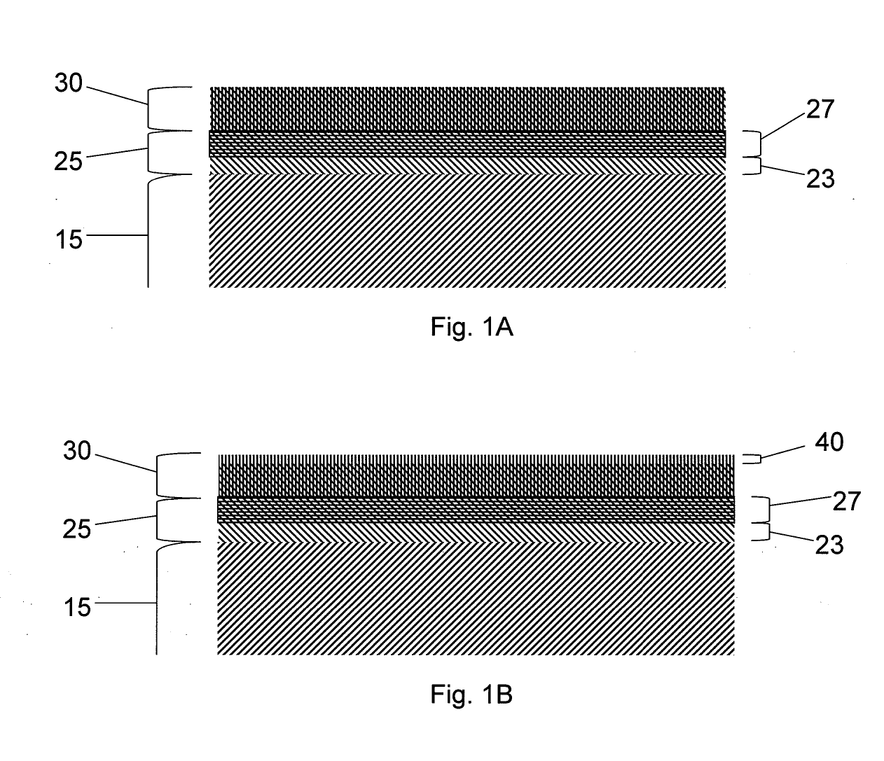

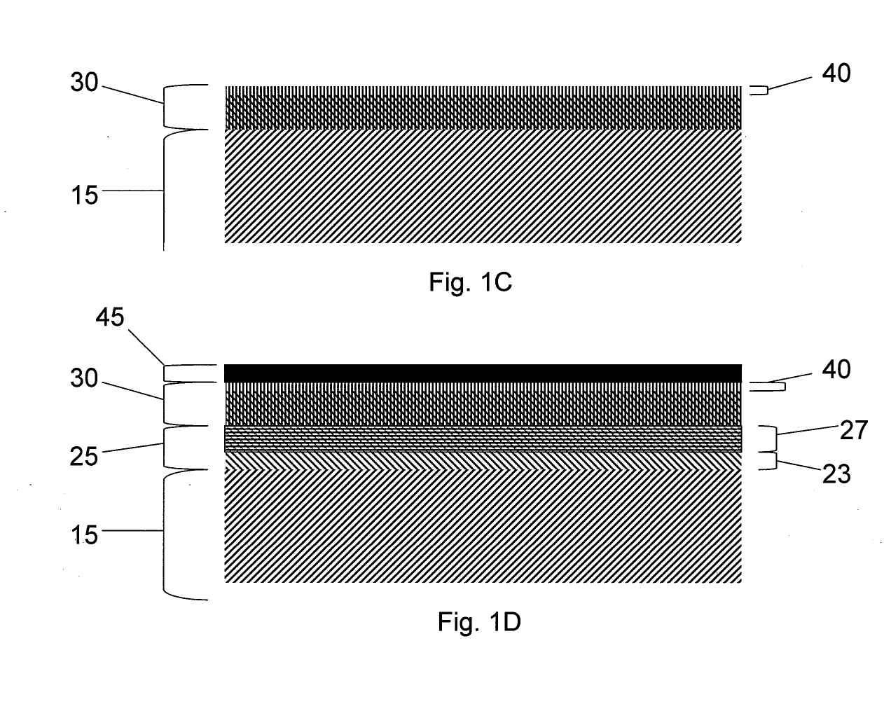

FIG. 1A is an illustrative cross-sectional view of surface layers on an implantable device prior to texturing in accordance with an embodiment of the invention. FIG. 1B is an illustrative cross-sectional view of surface layers on an implantable device after texturing in accordance with an embodiment of the invention. FIG. 1C is an illustrative cross-sectional view of surface layers on an implantable device having a coating over the textured surface in accordance with an embodiment of the invention.

Referring to FIG. 1A, an implantable device is shown with a surface layer 30, also referred to as a capping layer 30, of co-deposited atoms of at least two material types. In an embodiment, as shown in FIG. 1A, the surface layer 30 is on an adhesion layer 25, which in turn is on a substrate 15.

In an embodiment of the invention, the substrate material 15 can include materials such as cobalt-chromium, steel, stainless steel, nickel-based steel, titanium, and nitinol, and alloys thereof, or other materials that provide applicable properties, for example, material strength, flexibility and malleability, depending on the application. In an embodiment, the substrate 15 includes radiopaque materials that enable the implantable device to be detectable by radiography or fluoroscopy, for example, stainless steel, nickel-based steel, cobalt-chromium, titanium, nitinol, and alloys thereof.

The adhesion layer 25 comprises at least one of a first portion 23 and an optional second portion 27 that provides increased adhesion of layer 30. In an embodiment, the first portion 23 of adhesion layer 25 comprises adhesion layer materials that provide a strong bond to the substrate surface, such as palladium or gold. In an embodiment, the second portion 27 is between the first portion 23 and the surface layer 30, and comprises a gradated mixture of adhesion layer material, such as palladium or gold, and capping layer material, such as platinum, or another material which provides a substantially biocompatible surface and permits a strong bond between surface layer 30 and adhesion layer 25. In an embodiment, the surface layer 30 comprises the capping layer material including at least one of platinum, platinum-iridium, platinum-iridium, gold, nickel, steel, or alloys thereof.

Referring to FIG. 1C, another embodiment of the invention includes surface layer 30 directly on the substrate 15, wherein no adhesion layer 25 is present between the surface layer 30 and the substrate 15. For example, while FIGS. 1A-1B illustrate an adhesion layer 25 comprising both a base layer, or first portion 23, and a transition layer, or second portion 27, other applicable embodiments may include a surface layer 30, no adhesion layer, or no transition layer and/or no second portion 27, disposed between the substrate 15 and the surface layer 30, for example, similar to those embodiments described in U.S. Patent Application No. 60/895,924 filed on Mar. 20, 2007, entitled “Implantable Devices and Methods of Forming the Same,” incorporated herein by reference.

In an embodiment, the implantable device comprises a surface layer 30 having a thickness between 100 and 25,000 angstroms with a nano-textured finish. Other embodiments include capping layer thicknesses of between about 5,000 and 10,000 angstroms and an adhesion layer thickness of between about 500 and 2500 angstroms, or at least one of the capping layer and the adhesion layer having a thickness between about 100 and 5000 angstroms, or at least one of the capping layer and the adhesion layer having a thickness of less than about 2500 angstroms. Thicknesses of the capping layer depend in part on the shape of the substrate surface and the desired porosity of the surface. For instance, configurations having greater porosities will require a surface layer 30 of greater thickness in order to maintain its integrity (i.e. uniformity and adhesiveness). In other embodiments, the transition portion 27 of the adhesion layer 25 can be between a few atoms thick to about 2000 angstroms.

In an embodiment, the surface layer 30 comprises at least one of a first type of material and a second type of material. The first type of material of the surface layer 30 is resistant to a removal process and the second type of material is susceptible to the removal process. In an embodiment of the invention, the first type of material is a biocompatible metal for implantation into a human vessel such as, for example, platinum, platinum-iridium, gold, nickel, steel, or alloys thereof. In an embodiment, the second type of material can be any material that is not resistant to the electrochemical bath, including copper, chromium, silver, or alloys thereof.

In an embodiment, the removal process comprises an electrochemical bath. Agents that could be used for removal of the second type of material can include, for example, nitric acid, sulphuric acid, or phosphoric acid, or other materials depending on the types of deposited materials. Other methods and materials for use in the removal process are described in U.S. Pat. No. 6,979,346, incorporated herein by reference.

Referring to FIG. 1B, after the removal process is applied, the exterior of surface layer 30 comprises voids (or nucleation sites) where the atoms were once located, forming a textured outer surface 40. The typical (or average) size of the voids left by the removal process can be varied by controlling the ratio of the first and second types of material and will be dependent upon the size and bonding characteristics of the different atoms. For example, the span across an atom-sized void can be roughly estimated by known van der Waals radii, which are established from contact distances between non-bonding atoms in touching molecules or atoms and the densities of the deposited layers. The radii for platinum and silver bonds, for example, are about 2 angstroms, and the densities of the layers provided by embodiments of the invention can be between at least about 95% and 98% of full bulk density, thus providing void diameters potentially as small as 4 angstroms.

An embodiment of the invention provides a texturing that will promote healthy endothelial growth and reduces excessive occlusive growth such as, for example, excessive smooth muscle cell growth, thrombin, and/or fibrin. An embodiment of the invention for this purpose includes a surface texture factor (the root mean square of the breadth of the voids) of less than about 3 microns and preferably between about 0.01 microns and one micron. An embodiment of the invention includes a surface texture factor of between about 0.003 and 0.01 microns which may be preferable in certain cases where the thickness of the textured layer (e.g. surface layer 30) is of a thickness (e.g. less than 200 nanometers) where a minimal surface texture factor is preferred in order to maintain the integrity of the surface layer. Embodiments of the invention may also include texture factors adapted and balanced to provide for multiple functions (e.g., healthy growth, stent retention, and/or providing a therapy-delivering mechanism).

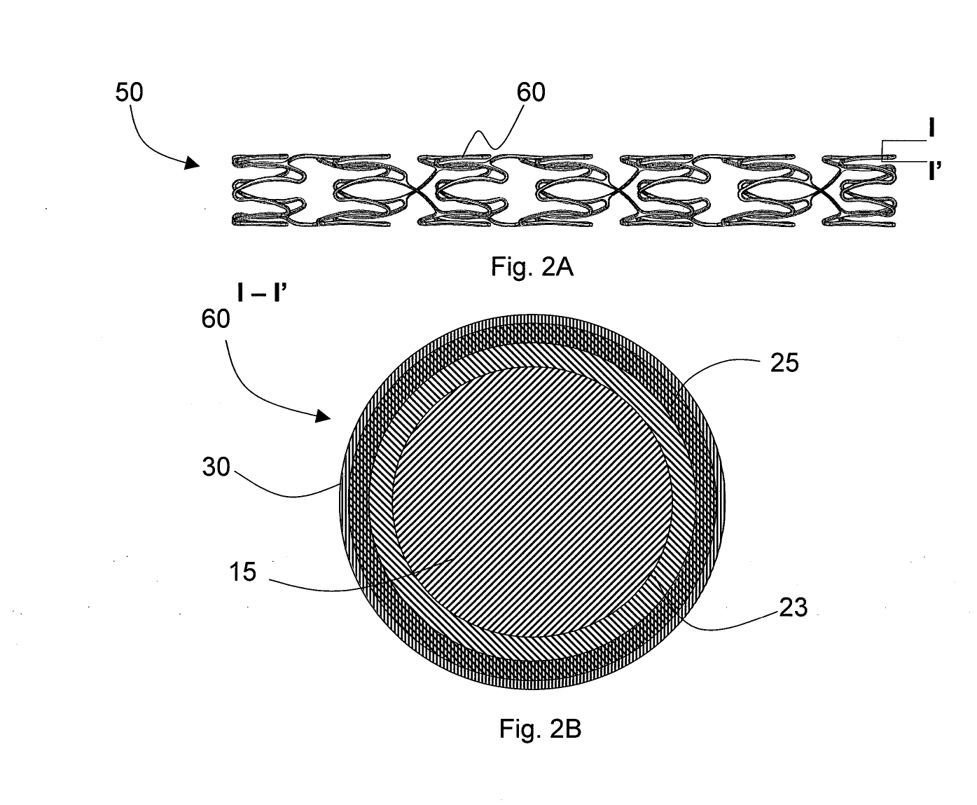

In embodiments of the invention, textured surface 40 can be adapted to elute drugs or other agents upon implantation. For example, U.S. Patent Publication No. US 2006/0271169 A1, incorporated herein by reference, describes the use of textured surfaces for containing and releasing therapeutic agents. In an embodiment of the invention, textured surface 40 can be adapted to provide a frictional surface between itself and a delivery system. In an embodiment of the invention, the textured surface 40 is adapted for use on a stent, for example, stent 50 shown in FIG. 2A below, wherein the surface 40 is textured (or roughened) to provide improved retention with a delivery system such as, for example, a balloon catheter (not shown). Degrees of roughness useful for these and similar purposes are described more fully in, for example, U.S. Pat. No. 6,979,346, the entire contents of which is herein incorporated by reference.

Referring to FIG. 1D, an illustrative cross-sectional view of surface layers on an implantable device having a coating 45 on the textured surface 40 is shown in accordance with an embodiment of the invention. In an embodiment of the invention, the textured surface 40 is designed to provide increased adhesion to a coating 45. In an embodiment, the coating 45 can be a polymer for eluting drugs or therapeutic agents that are applied as coatings to stents. Such eluting drugs or therapeutic agents are commonly employed and can include, for example, paclitaxel, rapamycin, and sirolimus, among others.

FIG. 2A is an illustrative side view of a stent in accordance with an embodiment of the invention. Referring to FIG. 2A, an illustrative perspective view of a stent 50 is shown having a substrate upon which at least one of an adhesion layer and capping layer may be sequentially deposited.

In an embodiment, a general relationship between layers deposited on a stent strut 60 is shown in FIG. 2B in a transverse cross-sectional illustrative view. In embodiments of the invention and in order to provide a strut 60 which is strong, expandable, and sufficient for retaining adequate radial forces after deployment, substrate material 15 may include steel, stainless steel, nickel-based steel, cobalt-chromium, titanium, nitinol, and alloys thereof.

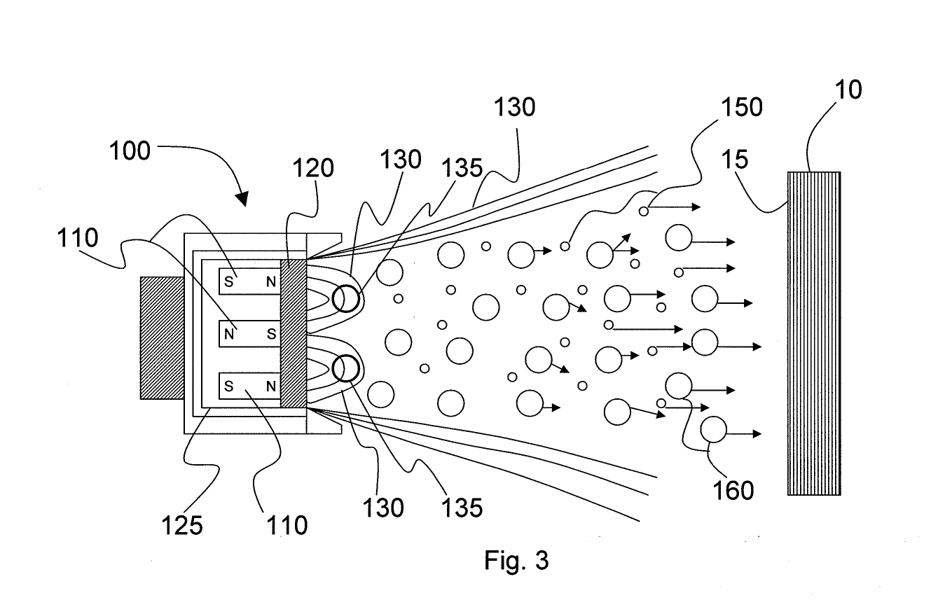

FIG. 3 is an illustrative view of an unbalanced magnetron depositing atoms onto a substrate for purposes of forming a textured surface in accordance with an embodiment of the invention. Referring to FIG. 3, a magnetron 100 is used initially to deposit atoms of the adhesive surface layers over the substrate surface 15, including, for example, the capping layer 30 or adhesion layer 25 of FIGS. 1A-1C, 2A-2B. In an aspect of the invention, the magnetron 100 is an unbalanced magnetic field magnetron. The magnetron 100 creates an unbalanced magnetic field 130, wherein a plasma cloud 135 of metal atoms 160 and bombarding noble ions 150 is produced in the unbalanced magnetic field 130. The metal atoms 160 and bombarding noble ions 150 are supplied from a source 120 which is positioned in front of a plurality of magnets 110 placed within a cooling block 125, which permits the magnetron 100 to create the unbalanced magnetic field 130. In this manner, the magnetron 100 can direct both the flux of metal atoms 160 and the flux of bombarding noble ions 150 onto the substrate 15 in a substantially collinear direction from the plasma cloud 135. As a result, the bombarding noble ions 150 impact and condense metal atoms 160, producing a substantially uniform layer of metal atoms on the substrate surface. Conventional balanced magnetic field magnetrons, on the other hand, generally depend on the use of independent plasma clouds of coating metal atoms and bombarding noble ions, and can subsequently produce an inconsistent coating. The general methods of use and embodiments of magnetron systems in accordance with embodiments of the invention are more fully described in U.S. Pat. No. 7,077,837, incorporated by reference herein.

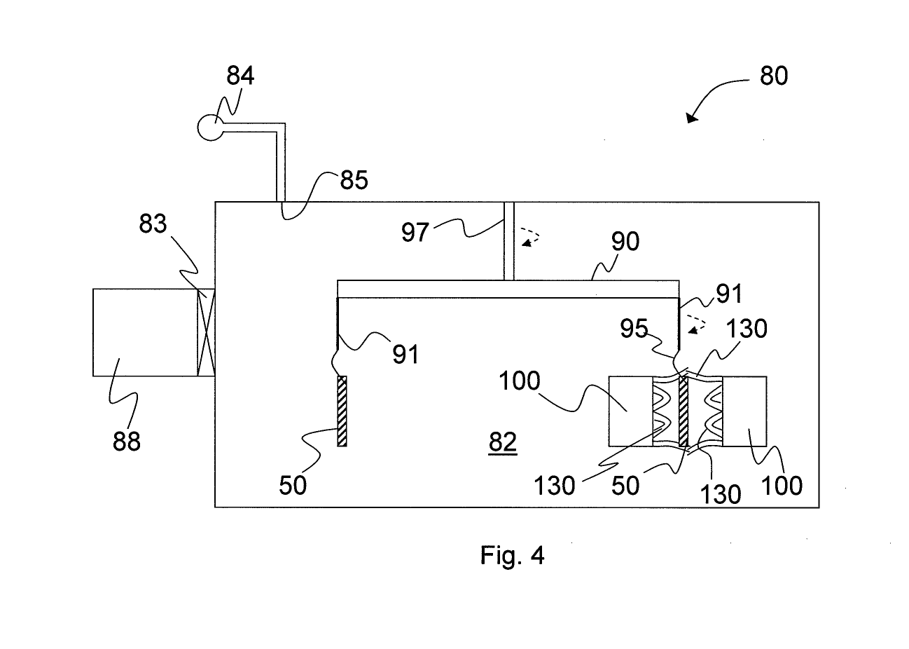

In order to form the surface layer 30 comprising at least two types of metallic atoms (e.g. of platinum and silver) and/or transition layer 27 (e.g. of platinum and palladium), for example, an embodiment of the invention simultaneously operates at least two unbalanced magnetrons 100 to generate a flux of each of the respective metals 160 and bombarding ions 150. Referring to FIG. 4, an illustrative side-perspective schematic of an apparatus 80 for coating a substrate is shown according to an embodiment of the invention. Two or more magnetrons 100 are positioned relative to each other so that they can simultaneously direct a flux of different metal atom types toward the substrate of a stent 50. As a stent 50 is held in place between the fluxes 130 of magnetrons 100 by a fixture 91, which rotates stent 50 as the different metal atoms are deposited, thereby creating a substantially uniform coating of atoms mixed among the types deposited by each of the magnetrons 100. In an embodiment of the invention, a flexible attachment 95 allows stent 10 to vibrate in a substantially random manner, thus promoting further uniformity of the deposited layers. In an embodiment of the invention for creating capping layer 30 of two types of atoms, one magnetron 100 of a two or more magnetron embodiment can deposit the atoms of a first type resistant to a removal process (e.g., platinum, platinum-iridium, or gold atoms) while a second magnetron 100 can deposit a second type of material not resistant to the removal process (e.g., copper, chromium, or silver). Simultaneous deposition with the use of multiple magnetrons is described in further detail in previously referenced U.S. Pat. No. 7,077,837 and U.S. Patent Application No. 60/895,924 filed on Mar. 20, 2007, entitled “Implantable Devices and Methods of Forming the Same,” incorporated herein by reference above.

The magnetrons 100 can be controlled in synchronization (e.g. with the use of a processor) to deposit desired ratios of each of the types of metals. The multiple magnetrons 100 may be controlled to maintain or change in-process the concentration ratio of atoms of one type with respect to the other type(s). For example, in an embodiment of the invention, the relative ratio of second material atoms susceptible to a removal process, e.g., silver atoms, to first material atoms not susceptible to a removal process, e.g., platinum atoms, in surface layer 30 can be gradually increased/decreased until a ratio is achieved for obtaining the desired texturing resolution in which voids are created by removal of the second material items from the surface 15 exterior. Preferable thicknesses and combinations of materials interfacing adhesion layer 25 and capping layer 30 with substrate 15 are more fully described in U.S. Patent Application No. 60/895,924 filed on Mar. 20, 2007, entitled “Implantable Devices and Methods of Forming the Same,” incorporated herein by reference above.

Further referring to FIG. 4, an apparatus 80 is provided for processing multiple stents in a batch process using one or more magnetrons. Fixture 91 holding a stent 50 is attached at one end to a wheel 90 which is rotatable and driven via an axle 97 and an actuating mechanism (not shown). After one stent 50 has been coated by magnetrons 100, another stent 50 attached to wheel 90 can be actuated into place between magnetrons 100. In an embodiment of the invention, numerous stents 50 can be similarly attached to wheel 90 and coated in an automated manner with the aid of a programmed processor (not shown) that actuates wheel 90 and controls magnetrons 100, among various other components. Wheel 90 and attached stents 50 and magnetrons 100 are contained in a vacuum chamber 82. A vacuum of, for example, between 1E-3 to 1E-9 torr can be drawn from chamber 82 using a vacuum pump 88. Vacuum pumping may thereafter be throttled by a valve 83 and noble gas, for instance, argon or xenon, may be introduced from a source 84 through a port 85 into chamber 82. The chamber 82 may continue to be filled with the noble gas to a pressure ranging from about 0.1 mtorr to about 100 mtorr. Next, an electrical charge of about −200 VDC to about −1000 VDC may be applied to stent 50 to rid its surface of oxides and other contaminants such as, for example, oxides that can develop on a cobalt-chromium or steel substrate during manufacture and affect the adhesiveness and safety of the device. This pre-cleaning process of the device may last from about 5 to about 60 minutes, depending on the initial cleanliness of a stent 50. Once the ion pre-cleaning process is completed, the coating process using multiple magnetrons 100 may begin such as in accordance with the details discussed above and in connection with U.S. Pat. No. 7,077,837 incorporated by reference above.

The methods described above can improve the bulk density and texture of coatings relative to using traditional IBAD (ion beam assisted deposition) which is limited to about a maximum density of between 92% to less than about 95% of full bulk density (wherein full bulk density is representative of a fully compacted non-porous material). In various embodiments of the invention, the unbalanced magnetrons can provide the above described coatings at between about 95% to 98% of full bulk density for the designated metal atoms, thus allowing for higher resolution surface textures than classical IBAD methods (discrete non-collinear ion beam deposition). Classical IBAD applications may employ fields of between about 0.8 keV to 1.5 keV. In embodiments of the invention, fields of between about 40 eV and 250 eV acting on ions supplied by a plasma cloud are directed to a target surface in substantially collinear fashion with the deposited metal atoms. Although such a field may provide less power per ion than do typical discrete ion-beam methods, the reduced energy fields of various embodiments of the present invention are applied over a relatively denser array of ions (in the plasma field) along with metal atoms, promoting greater uniformity in the thickness and density of the layers. The less energized ions are also less likely to cause back-sputtering (and failing to deposit on the surface coating) and can promote moderate movement and shifting of the deposited metal atoms, thus providing greater distribution, enhanced density, and uniformity of the layers.

The textured surface layers in accordance with embodiments of the invention can provide for improved endothelial healing about the surface after implantation, improved adhesiveness with additional outer layers, to provide voids out of which drugs or other therapeutic agents can be eluted, and/or to increase friction between the surface and a deployment system.

In embodiments of the invention, bio-active materials can be deposited into the textured structure for release upon implantation. Such materials can include anti-restenosis, anti-thrombosis, and anti-inflammatory agents including, for example, paclitaxel, rapamycin, sirolimus, and/or agents to promote healthy endothelial growth including dipyridamole, genes, stem cells, and other cells including live cells. The agent may be incorporated into the textured layer in various embodiments through, for example, liquid immersion, vacuum desiccation, high-pressure infusion or vapor loading. By controlling the ratio of the different types of atoms simultaneously deposited, the size of the voids in the metallic structure may be modified to subsequently control the rate and amount of release of the agent.

For providing a surface to facilitate re-endothelialization, a target surface texture or resolution of an embodiment of the invention can be made textured to promote healthy attachment of endothelial cells while avoiding an abundance of unhealthy occlusive cell attachment such as that of smooth muscle cells, platelets, thrombin, and fibrin that could result in, for example, restonosis or thrombosis. In embodiments of the invention, voids of the textured exterior provides a surface texture factor (the root mean square of the breadth of the voids) of preferably less than about a micron and at least less than about 3 microns, with embodiments also including surface texture factors of between about than 0.003 microns and 0.01 microns. Other surface texture factors are described in, for example, U.S. Pat. No. 6,517,571 by Brauker et al., incorporated herein by reference.

In another aspect of the present invention, a textured surface is provided on a polymer-based coating such as, for example, a polymer-based embodiment of coating layer 45 or on a surface of one of many commercially distributed polymer-coated implantable devices (e.g. the Taxus® stent distributed by Boston Scientific of Natick, Mass.). In an embodiment of the invention, a method is provided for texturing the surface of a polymer coating by bombarding the polymer surface with ions from an unbalanced magnetron used in embodiments of the invention previously described without the aspect of depositing metallic atoms on the surface. The lower energy field of embodiments of the present invention, e.g. between about 50 eV and 250 eV, as compared to traditional IBAD (e.g. 0.8 keV to 1.5 keV), reduces the likelihood of overheating and damage to the integrity of the polymer exterior. In an embodiment, a flux of bombarding ions such as, for example, argon or xenon ions, are generated and directed toward the polymer surface, creating voids or cavities in the surface in a substantially uniform manner. The atomic diameter of xenon ions, for example, can be about 1.64 angstroms. Over time, the deposited ions will create voids at the sub-nanometer level and, over increasing time, voids of larger sizes in a relatively uniform manner as the unbalanced magnetron directs the ions at generally evenly distributed locations on the polymer surface. Accordingly, in embodiments of the invention, the polymer surface has a surface texture factor of less than about a micron and, in another embodiment of the invention, a surface texture factor of less than about a nanometer.

In embodiments of the invention, the textured polymer surface can be adapted for use in similar ways as that of the textured metallic surfaces of previously described embodiments. For example, the textured surface can provide for improved endothelial healing about the surface after implantation, improved adhesiveness with additional outer layers, to provide voids or pores out of which drugs or other therapeutic agents can be eluted, or to increase friction between the surface and a deployment system.

It will be understood by those with knowledge in related fields that uses of alternate or varied materials and modifications to the methods disclosed are apparent. This disclosure, including the claims herein, are intended to cover these and other variations, uses, or other departures from the specific embodiments as come within the art to which the invention pertains.

1. A method for coating an implantable device comprising the steps of:
providing a substrate surface of an implantable device;
forming a coating layer on the substrate surface by directing a mixture of atoms of a first type of material and of a second type of material toward the substrate surface while simultaneously and substantially collinearly bombarding the substrate surface with ions, wherein the first type of material is substantially resistant to a removal process and the second type of material is substantially susceptible to a removal process; and
creating voids in the coating layer to form a textured exterior on the coating layer by applying the removal process to selectively remove at least some of the atoms of the second type of material from the coating layer surface.

2. The method of claim 1 wherein said first type of material comprises at least one of platinum, platinum-iridium, gold, and alloys thereof.

3. The method of claim 2 wherein the second type of material comprises at least one of copper, chromium, silver, and alloys thereof.

4. The method of claim 1 wherein the removal process comprises exposing the coating layer to an electrochemical bath.

5. The method of claim 1 wherein the removal process comprises applying an acid in which the first type of material substantially detaches from the surface of the coating layer and in which said second type of material does not substantially detach from the coating layer.

6. The method of claim 5 wherein said acid comprises at least one of at least sulphuric acid, nitric acid, or phosphoric acid.

7. The method of claim 1 wherein voids of said textured exterior provides a surface texture factor of less than about 3 microns.

8. The method of claim 7 wherein said textured exterior has a surface texture factor of less than about 1 micron.

9. The method of claim 8 wherein said textured exterior has a surface texture factor of less than about 0.1 microns.

10. The method of claim 9 wherein said textured exterior has a surface texture factor of less than about 0.01 microns.

11. The method of claim 10 wherein said textured exterior has a surface texture factor of between about 0.003 and 0.01 microns.

12. The method of claim 1 wherein the deposited mixture of atoms of the first and second types are substantially of a density of greater than about 95% full bulk density.

13. The method of claim 1 further comprising the step of coating the textured exterior with a polymer.

14. The method of claim 1 further comprising the step of infusing the voids of the textured exterior with a therapeutic agent.

15. The method of claim 14 wherein the therapeutic agent is at least one of paclitaxel, rapamycin, sirolimus, genes, or stem cells.

16. An implantable device comprising:
a substrate;
one or more coatings on the substrate, the coatings comprising a textured layer of metal atoms deposited onto the device by a method of deposition with substantially collinear ion-bombardment, the textured layer of metal atoms having a surface factor of less than about 3 microns.

17. The implantable device of claim 16 wherein the exterior of said textured layer has a surface texture factor of less than about 1 micron.

18. The implantable device of claim 17 wherein the exterior of said textured exterior has a surface texture factor of less than about 0.1 microns.

19. The implantable device of claim 18 wherein the exterior of said textured layer has a surface texture factor of less than about 0.01 microns.

20. The implantable device of claim 19 wherein said porous exterior has a surface texture factor of between about 0.001 and 0.01 microns.

21. The implantable device of claim 19 wherein said porous exterior has a surface texture factor of between about 0.0004 and 0.001 microns.

22. The implantable device of claim 16 wherein at least one or more of said coatings is substantially of a density of greater than about 95% full bulk density.

23. The implantable device of claim 16 further comprising a polymer layer adjacent said textured layer.

24. The implantable device of claim 16 further comprising a therapeutic agent infused into voids of said textured layer.

25. The implantable device of claim 24 wherein said therapeutic agent is at least one of paclitaxel, rapamycin, sirolimus, dipyridamole, genes, stem cells, and combinations thereof.

26. A medical stent comprising a textured surface with a surface texture factor of less than about 0.01 microns.

27. The medical stent of claim 26 wherein said surface texture factor is between about 0.0004 and 0.01 microns.

28. A method for providing a textured polymer surface of an implantable device comprising the steps of:
providing a polymer surface;
generating a flux of bombarding ions; and
directing the ions toward the polymer surface in order to form a plurality of substantially uniformly distributed voids on the polymer surface.

29. The method of claim 28 wherein said ions are at least one of xenon or argon ions.

30. The method of claim 29 wherein the voids provide a surface texture factor of less than about a micron.

31. The method of claim 30 wherein the voids provide a surface texture factor of less than about a nanometer.

32. An implantable device comprising a textured polymer surface formed by the method of claim 28.

33. An implantable device comprising a textured polymer surface having a surface texture factor of less than about 3 microns.

34. An implantable device comprising a textured polymer surface having a surface texture factor of less than about a micron.

35. The method of claim 1 wherein an adhesion layer is formed on the substrate before forming said coating layer, the adhesion layer comprising at least one portion substantially comprising palladium.

36. The implantable device of claim 16 further comprising an adhesion layer on the substrate, the adhesion layer comprising at least one portion substantially comprising palladium.
